(19) United States
(12) Patent Application Publication
(10) Pub. No.: US 2009/0098655 A1
(43) Pub. Date: Apr. 16, 2009

(54) Detection Apparatus and Methods

(75) Inventors: John Leslie Brokenshire, Torrington (GB); Basil Polychronopulos, Luton (GB)

Correspondence Address: FOLEY AND LARDNER LLP;SUITE 500, 3000 K STREET NW, WASHINGTON, DC, 20007, US

(73) Assignee: Smiths Detection-Watford Limited

(21) Appl. No.: 12/225,481

(22) PCT Filed: Apr. 4, 2007
(86) PCT No.: PCT/GB2007/001251

§ 371 Date: Sep. 23, 2008

(30) Foreign Application Priority Data
Apr. 13, 2006 (GB) 0607470.2

(51) Int. Cl. G01N 33/20; G01N 33/00; B01J 19/00

(52) U.S. Cl. 436/73; 436/103; 422/83

(57) ABSTRACT

A chemical cell (40) containing silver nitrate is connected at the inlet (10) of an ion mobility spectrometer (1) so that sample gas for detection flows to the IMS via the chemical cell. When the sample gas contains arsine or phosphine, to which the IMS is not normally sensitive, this is converted by the silver nitrate in the cell (40) to nitric acid, to which the IMS is sensitive. In order to distinguish between nitric acid produced in response to arsine or phosphine and nitric acid present in the sample gas before supply to the cell (40), the inlet (10) is switched to receive sample gas directly.

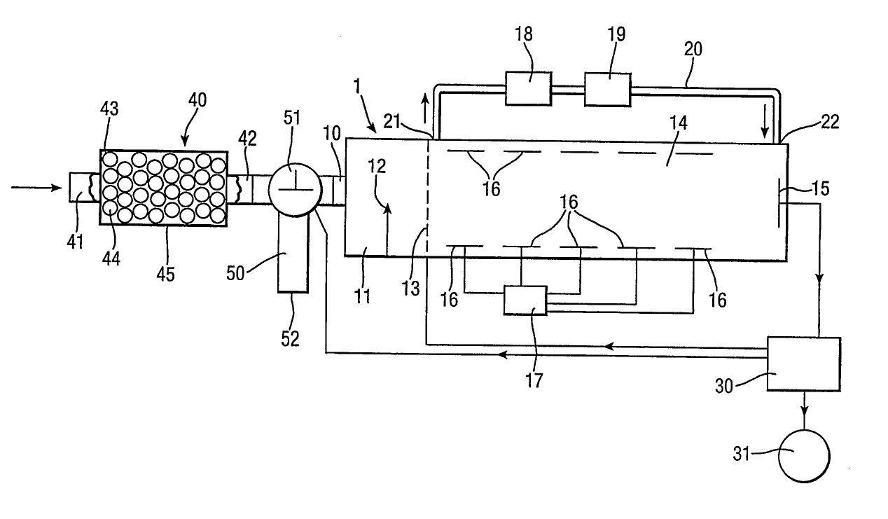

This invention relates to detection apparatus of the kind for detecting the presence of arsine or phosphine in a sample gas, the apparatus including a detection unit having a gas inlet, the detection unit being relatively unresponsive to the presence of arsine or phosphine within the unit.

Ion mobility spectrometers are commonly used to detect the presence of, and indicate the nature of, hazardous substances in air. IMSs work effectively for a wide range of hazardous substances but certain configurations of IMS are not able reliably to detect the presence of arsine or phosphine because these substances do not form detectable product ions in an IMS detector. Arsine (or AsH₃, arsine gas, arsine exposure, arseniuretted hydrogen, arsenous hydride, arsenic trihydride, hydrogen arsenide) and phosphine (or phosphorus hydride, PH₃ or phosphane) are highly toxic chemicals, which could be used as terrorist or military chemical weapons. It is, therefore, important to be able to detect their presence at low levels in a reliable manner, preferably using a detector that could also be used to detect other hazardous substances.

It is an object of the present invention to provide alternative detection apparatus and methods.

According to one aspect of the present invention there is provided detection apparatus of the above-specified kind, characterised in that the apparatus includes a chemical cell having an inlet connected to receive the sample gas and an outlet connected with the gas inlet of the detection unit, and that the chemical cell is operative to produce a chemical that can be detected more readily by the detection unit when arsine or phosphine is supplied to the cell.

The apparatus is preferably arranged to supply the sample gas to the detection unit independently of the chemical cell when the apparatus detects the presence of the chemical such as to determine whether the chemical is present in the sample gas before passage through the chemical cell. The chemical cell is preferably arranged to produce nitric acid in response to the presence of arsine or phosphine. The chemical cell preferably includes silver nitrate, which may be coated on a surface within the cell, such as on granules in the cell. The detection unit preferably includes an ion mobility spectrometer.

According to another aspect of the present invention there is provided a chemical cell for use in detection apparatus according to the above one aspect of the present invention.

According to a further aspect of the present invention there is provided a chemical cell having an inlet and an outlet, characterised in that the outlet is adapted for connection to a detection unit, that the inlet is adapted to receive sample gas containing suspected arsine or phosphine, and that the cell contains silver nitrate effective to produce nitric acid in response to passage of arsine or phosphine through the cell.

According to a fourth aspect of the present invention there is provided a method of detecting the presence of arsine or phosphine in a sample gas, characterised in that the method includes the steps of supplying the sample gas to contact a chemical substance arranged to produce in the presence of arsine or phosphine a second chemical different from arsine or phosphine, detecting the presence of the second chemical and providing an output response indicative of the detection of arsine or phosphine.

The second chemical is preferably nitric acid. The method preferably includes the step of by-passing the chemical substance when the presence of the second chemical is indicated, such as to determine whether the second chemical is present in the sample gas before passage through the chemical cell. The chemical substance preferably contains silver nitrate.

According to a fifth aspect of the present invention there is provided apparatus for use in a method according to the above fourth aspect of the present invention.

Ion mobility spectrometer apparatus and its method of operation, according to the present invention, will now be described, by way of example, with reference to the accompanying drawing which is a schematic, cross-sectional side elevation view of the apparatus.

The apparatus includes a conventional ion mobility spectrometer unit 1 having an inlet 10 by which a sample gas is supplied to the interior of the unit for detection and identification. The inlet 10 opens at the left-hand end of the unit 1 into an ionization and reaction region 11 including a corona discharge needle 12 or other arrangement for ionizing the substances within the sample gas. An electrical shutter 13 isolates the ionization region 11 from a drift chamber 14 having a collector plate 15 at its far end, remote from the shutter. Electrodes 16 are spaced along the drift chamber 14 and are connected to a voltage source 17 so that an electrical field can be established along the drift chamber to cause ions admitted by the shutter 13 to move from left to right towards the collector plate 15. A pump 18 and molecular sieve 19 are connected in a gas flow path 20 extending from an inlet 21 towards the left-hand end of the unit 1 to an outlet 22 towards the right-hand end of the unit so that cleaned and dried gas is flowed along the drift chamber 14 from right to left, against the ion flow, in the usual way. The collector plate 15 is connected to a processor 30, which is also connected to control the shutter 13. The processor 30 detects the charge produced when an ion or ion cluster hits the collector plate 15 and computes the time of flight. From this information the nature of many ion species can be identified and an output provided to utilization means, such as a display 31.

As so far described, the apparatus is conventional and suffers from an inability to detect arsine or phosphine.

The apparatus is modified by the inclusion of a chemical cell 40 having an inlet 41 open to atmosphere or to some other source of sample gas. The outlet 42 of the cell 40 is connected to the inlet 10 of the spectrometer unit 1 so that all gas supplied to the spectrometer inlet first passes through the cell. The cell 40 contains a chemical 43 effective to produce a second chemical in the presence of arsine or phosphine, the second chemical being different from arsine or phosphine and being one that can be detected reliably by the ion mobility spectrometer 1. More particularly, the chemical 43 in the cell 40 is silver nitrate, which reacts with arsine and phosphine to produce nitric acid vapour, which is readily detected in the IMS 1. One molecule of arsine, for example, produces six molecules of nitric acid (Demange, Elcabache et al, J Environ Monit 2000, October 2(5), 476-82). It can be seen, therefore, that, even if the IMS were capable of detecting arsine by itself, the use of silver nitrate would result in a possible six-fold improvement in sensitivity. The chemical 43 may be contained within the cell 40 in various different ways. For example, as shown, it may be coated on the surface of granules 44 packed into a cylindrical housing 45 so that a large area is exposed to gas flowing along the cell. Alternatively, the chemical could be in powder form, or it could be coated on a mesh or fibrous material or it could simply be provided as a lining on the walls of the cell. The silver nitrate does not itself vaporize at normal temperatures or otherwise cause a response in the IMS 1.

It can be seen, therefore, that sample gas supplied to the IMS unit 1 must flow through the chemical cell 40. The IMS unit 1 responds in the normal way to the usual range of substances to which it is responsive. If arsine or phosphine were present in the sample gas, some at least of this would be converted in the cell 40 to nitric acid vapour which would flow to the IMS inlet 10 for detection. The output response produced by the processor 30 would be the same as that for nitric acid. The processor 30 could be arranged to provide an indication on the display 31 of the legend “Arsine/Phosphine” if the apparatus were used in situations where arsine or phosphine was more likely than nitric acid. Alternatively, it could be arranged to provide a display indication “Arsine/Phosphine or Nitric Acid”.

In order to resolve the ambiguity between the response to the two chemicals, the apparatus preferably includes a by-pass inlet path 50 connected to the IMS inlet 10, that is, between the IMS unit 1 and the chemical cell 40 via a three-way valve 51. The by-pass inlet path 50 has an inlet 52 open to atmosphere or to the source of the sample gas being detected. The valve 51 is controlled by the processor 30 and its normal position, as shown, is with the chemical cell outlet 42 in gas-flow connection with the IMS inlet 10 and with the by-pass inlet 52 isolated. When the processor 30 detects the presence of nitric acid, it switches the valve 51 to isolate the chemical cell 40 from the IMS inlet 10 and to connect instead the by-pass inlet 52 to the IMS unit 1 so that air or sample gas is supplied directly to the IMS inlet 10 without passing through the chemical cell 40. If the IMS unit 1 still produces the same response then it is clear that the nitric acid is present in the air or sample gas itself and is not produced by the chemical cell 40. The display 31, therefore, produces a response “Nitric Acid”. However, if the nitric acid response were to cease when the valve 51 was switched to the bypass position, it would be clear that all the nitric acid was being produced by the chemical cell 40 in response to arsine or phosphine, so the processor 30 would cause the display 31 to indicate “Arsine/Phosphine”. If the response did not disappear completely but fell in magnitude when switched to the by-pass state, this could indicate the presence of both nitric acid and either arsine or phosphine so the display 31 would be caused to show “Arsine/Phosphine & Nitric Acid”.

It is possible that other nitrate-containing chemicals could be used instead of silver nitrate to produce nitric acid when exposed to arsine or phosphine. Similarly, there might be other chemicals with which arsine and phosphine would react to produce a second chemical other than nitric acid and that is detectable in a detector. The use of silver nitrate or the like could even have application in detection apparatus that is capable of detecting arsine and phosphine because of the six-fold improvement in sensitivity the reaction produces, as mentioned above.

Although the invention is suitable for use in an ion mobility spectrometer it is possible that it could be used in other forms of detector such as mass spectrometers and electrochemical and conductivity detectors.

1. Detection apparatus for detecting the presence of arsine or phosphine in a sample gas, the apparatus including a detection unit having a gas inlet, the detection unit being relatively unresponsive to the presence of arsine or phosphine within the unit, wherein the apparatus includes a chemical cell having an inlet connected to receive the sample gas and an outlet connected with the gas inlet of the detection unit, and that the chemical cell is operative to produce a chemical that can be detected more readily by the detection unit when arsine or phosphine is supplied to the cell.

2. Detection apparatus according to claim 1, wherein the apparatus is arranged to supply the sample gas to the detection unit independently of the chemical cell when the apparatus detects the presence of the chemical such as to determine whether the chemical is present in the sample gas before passage through the chemical cell.

3. Detection apparatus according to claim 1, wherein chemical cell is arranged to produce nitric acid in response to the presence of arsine or phosphine.

4. Detection apparatus according to claim 1, wherein the chemical cell includes silver nitrate.

5. Detection apparatus according to claim 4, wherein the silver nitrate is coated on a surface within the cell.

6. Detection apparatus according to claim 5, wherein the silver nitrate is coated on granules in the cell.

7. Detection apparatus according to claim 1, wherein the detection unit includes an ion mobility spectrometer.

8. A chemical cell for use in detection apparatus according to claim 1.

9. A chemical cell having an inlet and an outlet, wherein the outlet is adapted for connection to a detection unit, that the inlet is adapted to receive sample gas containing suspected arsine or phosphine, and that the cell contains silver nitrate effective to produce nitric acid in response to passage of arsine or phosphine through the cell.

10. A method of detecting the presence of arsine or phosphine in a sample gas, wherein the method includes the steps of supplying the sample gas to contact a chemical substance arranged to produce in the presence of arsine or phosphine a second chemical different from arsine or phosphine, detecting the presence of the second chemical and providing an output response indicative of the detection of arsine or phosphine.

11. A method according to claim 10, wherein the second chemical is nitric acid.

12. A method according to claim 10, wherein the method includes the step of by-passing the chemical substance when the presence of the second chemical is indicated, such as to determine whether the second chemical is present in the sample gas before passage through the chemical substance.

13. A method according to claim 10, wherein the chemical substance contains silver nitrate.

14. Apparatus for use in a method according to claim 10.
